(19) United States
(12) Patent Application Publication
(10) Pub. No.: US 2026/0102358 A1
(43) Pub. Date: Apr. 16, 2026

(54) METHODS FOR MAINTAINING CONCENTRATIONS OF VOLATILE LIPOPHILIC COMPOUNDS IN MANUFACTURE OF COMPOSITIONS AND DOSAGE FORMS

(71) Applicant: Curio IP, LLC, Towson, MD (US)

(72) Inventors: Amanda Karwic, Elverson, PA (US); Donald J. Treacy, JR., Woodbine, MD (US); Bruce Artman, Eldersburg, MD (US); Edward M. Rudnic, Georgetown, TX (US)

(21) Appl. No.: 19/421,506

(22) Filed: Dec. 16, 2025

Related U.S. Application Data
(63) Continuation of application No. 18/456,758, filed on Aug. 28, 2023, now abandoned.
(60) Provisional application No. 63/373,801, filed on Aug. 29, 2022.

(51) Int. Cl. A61K 31/01; A61K 9/00; A61K 31/015; A61K 31/045; A61K 47/12; A61K 47/14; A61K 47/24; A61K 47/26; A61K 47/36; A61K 47/38; A61K 47/42; A61K 47/44; A61K 47/46

(52) U.S. Cl. CPC A61K 31/01; A61K 9/0056; A61K 31/015; A61K 31/045; A61K 47/12; A61K 47/14; A61K 47/24; A61K 47/26; A61K 47/36; A61K 47/38; A61K 47/42; A61K 47/44; A61K 47/46

(57) ABSTRACT

Compositions and dosage forms that contain volatile lipophilic compounds, and methods of manufacturing such compositions and dosage forms, are described.

### CROSS-REFERENCE TO RELATED APPLICATIONS

This application is a Continuation of U.S. patent application Ser. No. 18/456,758, filed Aug. 28, 2023, which claims the benefit under 35 U.S.C. § 119(e) of U.S. Provisional Application No. 63/373,801, filed Aug. 29, 2022, the contents of which are incorporated by reference herein in their entirety.

### TECHNICAL FIELD

The present disclosure relates to methods of manufacturing compositions and dosage forms comprising volatile lipophilic compounds. The dosage forms may include, without limitation, solid dosage forms, chewable dosage forms, multi-particulate solids, fast dissolving dosage forms, semi-solid dosage forms, and inhaled dosage forms, among others. The present disclosure also relates to compositions and dosage forms provided according to the methods described herein.

### BACKGROUND

Volatile lipophilic compounds, such as essential oils, are substances that impart flavor, aroma, and in certain instances a therapeutic effect. However, because lipophilic compounds are often volatile, loss of the volatile lipophilic compounds during manufacture of various compositions and dosage forms is a significant manufacturing problem. However, development of improved methods of manufacturing compositions and dosage forms containing volatile lipophilic compounds is challenging.

### SUMMARY

According to a first aspect, the present disclosure relates in several embodiments to methods of providing a composition or a dosage form comprising one or more volatile lipophilic compounds. The method comprises: combining the one or more volatile lipophilic compounds with one or more water-insoluble components to provide a mixture; and processing the mixture to provide the composition or the dosage form, wherein the processing comprises one or more manufacturing methods capable of increasing vapor pressure of the one or more volatile lipophilic compounds when the one or more volatile lipophilic compounds are not combined with the one or more water-insoluble components.

The method may include the following details, which can be combined with one another in any combinations unless clearly mutually exclusive:
(i) the one or more volatile lipophilic compounds may have a first vapor pressure at a first temperature; the mixture may have a second vapor pressure at the first temperature; and the second vapor pressure may be lower than the first vapor pressure;
(ii) the method may further comprise: before the processing, adding one or more surface-active agents and one or more water-soluble components to the mixture; and emulsifying the mixture comprising the one or more surface-active agents and the one or more water-soluble components to provide an emulsion;
(iii) the one or more volatile lipophilic compounds may have a first vapor pressure at a first temperature; the emulsion may have a third vapor pressure at the first temperature; and the third vapor pressure may be lower than the first vapor pressure;
(iv) the one or more volatile lipophilic compounds may comprise one or more essential oils;
(v) the one or more essential oils may be selected from the group consisting of: myrcene, beta-caryophyllene, limonene, linalool, pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, cymene, octanol, isopulegol, cedrene, camphene, geranyl acetate, bergamotene, camphor, pulegone, and any combinations thereof;
(vi) the one or more water-insoluble components may comprise one or more oils, fats, waxes, polymers, or any combinations thereof;
(vii) the one or more water-insoluble components may be selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether, and any combinations thereof;
(viii) the one or more surface-active agents may be selected from the group consisting of: glyceryl monostearate, lecithin, a Tween, a Span, a modified food starch, a monoglyceride, a diglyceride, a cellulose derivative, guar gum, carrageenan, Acacia gum, a hydrocolloid, a fatty ester, a long-chain fatty acid, sodium citrate, pectin, and any combinations thereof;
(ix) the one or more water-soluble components may be selected from the group consisting of: gelatin, whey, casein, albumin, pectin, modified food starch, microcrystalline cellulose, hydroxypropyl methylcellulose, hydroxypropyl cellulose, polyvinyl alcohol, polyvinylpolypyrrolidone, beta celluloses, and any combinations thereof;
(x) the emulsifying may be performed using a high shear mixer;
(xi) the one or more manufacturing methods may be selected from the group consisting of: mixing, shearing, granulating, blending, heating, pressurizing, spray-drying, extrusion, spheronization, micro-encapsulation, and any combinations thereof;
(xii) the dosage form may be selected from the group consisting of: a solid dosage form, a soft chewable dosage form, a multi-particulate solid, a fast dissolving dosage form, a semi-solid dosage form, an inhaled dosage form, and any combinations thereof;
(xiii) the solid dosage form may be a compressed tablet, a lozenge, a troche, a chewable tablet, a capsule, a mini-tablet, or any combinations thereof;
(xiv) the soft chewable dosage form may be a gummy, a gum, or a combination thereof;
(xv) the multi-particulate solid may be a powder, a sprinkle, a suspension, a bulk granulation, or any combinations thereof;
(xvi) the fast dissolving dosage form may be a fast dissolving tablet, a fast-dissolving film, a sublingual tablet, a sublingual film, a buccal tablet, a buccal film, or any combinations thereof;
(xvii) the semi-solid dosage form may be a cream, an ointment, a tincture, a spray, a suppository, a liniment, a lotion, a gel, a paste, a balm, a salve, an infused bandage, an infused patch, or any combinations thereof;
(xviii) the inhaled dosage form may be a dosage form administrable with an aerosol inhaler, a vape pen, a vape device, and aerosol forming device, a dry powder inhaler, a nebulizer, or any combinations thereof;
(xix) the inhaled dosage form may be administrable by smoking and inhaling, wherein the smoking is by combustion, electronic means, or a combination thereof;
(xx) the gum may comprise one or more gum-forming components;
(xxi) the one or more gum-forming components may be selected from the group consisting of: gelatin, xanthan gum, guar gum, pectin, modified starch, and any combinations thereof;
(xxii) the gum may be formed using one or more methods selected from: tableting, molding, rolling, depositing, slabbing, or any combinations thereof;
(xxiii) the method may further comprise: after the emulsifying, covering the emulsion with a layer of one or more water-insoluble components;
(xxiv) the one or more surface-active agents may comprise one or more proteinaceous materials;
(xxv) the one or more proteinaceous materials may be selected from the group consisting of: albumin, gelatin, whey protein, a caseinate, a plant-based protein, and any combinations thereof.
(xxvi) the one or more volatile lipophilic compounds may comprise one or more terpenes.

According to a second aspect, the present disclosure also relates to compositions and dosage forms produced according to any of the methods described herein.

According to a third aspect, the present disclosure relates to a composition. The compositions comprises a mixture of one or more volatile lipophilic compounds and one or more water-insoluble components. The one or more volatile lipophilic compounds are present in an amount from 0.001% to 15% by weight of the composition.

The composition may include the following details, which can be combined with one another in any combinations unless clearly mutually exclusive:
(i) the one or more volatile lipophilic compounds may have a first vapor pressure at a first temperature; the mixture may have a second vapor pressure at the first temperature; and the second vapor pressure may be lower than the first vapor pressure;
(ii) the composition may comprise an emulsion of the mixture and one or more surface-active agents and one or more water-soluble components;
(iii) the emulsion may have a third vapor pressure at the first temperature, and the third vapor pressure may be lower than the first vapor pressure;
(iv) the one or more volatile lipophilic compounds may comprise one or more essential oils;
(v) the one or more essential oils may be selected from the group consisting of: myrcene, beta-caryophyllene, limonene, linalool, alpha-pinene, beta-pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, cymene, octanol, isopulegol, cedrene, camphene, geranyl acetate, bergamotene, camphor, pulegone, and any combinations thereof;
(vi) the one or more water-insoluble components may comprise one or more oils, fats, waxes, polymers, or any combinations thereof;
(vii) the one or more water-insoluble components are selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether, and any combinations thereof;
(viii) the one or more surface-active agents may be selected from the group consisting of: glyceryl monostearate, lecithin, a Tween, a Span, a modified food starch, a monoglyceride, a diglyceride, a cellulose derivative, guar gum, carrageenan, Acacia gum, a hydrocolloid, a fatty ester, a long-chain fatty acid, sodium citrate, pectin, and any combinations thereof;
(ix) the one or more water-soluble components may be selected from the group consisting of: gelatin, whey, casein, albumin, pectin, modified food starch, microcrystalline cellulose, hydroxypropyl methylcellulose, hydroxypropyl cellulose, polyvinyl alcohol, polyvinylpolypyrrolidone, beta celluloses, and any combinations thereof;
(x) the composition may be formulated as a dosage form selected from the group consisting of: a solid dosage form, a soft chewable dosage form, a multi-particulate solid, a fast dissolving dosage form, a semi-solid dosage form, and an inhaled dosage form;
(xi) the solid dosage form may be a compressed tablet, a lozenge, a troche, a chewable tablet, a capsule, a mini-tablet, or any combinations thereof;
(xii) the soft chewable dosage form may be a gummy, a gum, or a combination thereof;
(xiii) the multi-particulate solid may be a powder, a sprinkle, a suspension, a bulk granulation, or any combinations thereof;
(xiv) the fast-dissolving dosage form may be a fast-dissolving tablet, a fast-dissolving film, a sublingual tablet, a sublingual film, a buccal tablet, a buccal film, or any combinations thereof;
(xv) the semi-solid dosage form may be a cream, an ointment, a tincture, a spray, a suppository, a liniment, a lotion, a gel, a paste, a balm, a salve, an infused bandage, an infused patch, or any combinations thereof;
(xvi) the inhaled dosage form may be a dosage form administrable with an aerosol inhaler, a vape pen, a vape device, and aerosol forming device, a dry powder inhaler, a nebulizer, or any combinations thereof;
(xvii) the inhaled dosage form may be administrable by smoking and inhaling, wherein the smoking is by combustion, electronic means, or a combination thereof;
(xviii) the gum may comprise one or more gum-forming components;
(xix) the one or more gum-forming components may be selected from the group consisting of: gelatin, xanthan gum, guar gum, pectin, modified starch, and any combinations thereof;
(xx) the composition may further comprise a layer of one or more water-insoluble components disposed around the emulsion;
(xxi) the one or more water-insoluble components may comprise one or more oils, fats, waxes, polymers, or any combinations thereof;
(xxii) the one or more water-insoluble components may be selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether and any combinations thereof;
(xxiii) the one or more surface-active agents may comprise one or more proteinaceous materials;
(xxiv) the one or more proteinaceous materials may be selected from the group consisting of: albumin, gelatin, whey protein, a caseinate, a plant-based protein, and any combinations thereof;
(xxv) the total amount of the volatile lipophilic compounds may be present in an amount from 0.001% to 15% by weight of the composition;
(xxvi) at least one of the volatile lipophilic compounds may be present in an amount from 0.001% to 15% by weight of the composition.
(xxvii) the one or more volatile lipophilic compounds may comprise one or more terpenes.

### DETAILED DESCRIPTION

The present disclosure relates to methods of providing various mixtures, emulsions and dosage forms comprising one or more volatile lipophilic compounds. In various embodiments described herein, the methods described herein provide mixtures, emulsions and dosage forms that can at least partially stabilize the one or more volatile lipophilic compounds comprised in the mixtures, emulsions and/or dosage forms, and thereby minimize or prevent loss of the volatile lipophilic compounds during manufacturing processes using the mixtures, emulsions, and/or dosage forms.

In various embodiments, volatile lipophilic compounds are substances that impart flavor, smell or aroma, and in certain instances a therapeutic effect. These lipophilic compounds may be of natural origin or be synthetic, or a combination of both natural origin or synthetic. In some embodiments, the volatile lipophilic compounds can be essential oils. In some embodiments, the volatile lipophilic compounds may be derived from cannabis and/or hemp. In various embodiments, because these lipophilic compounds are volatile, they tend to be unstable during various dosage form manufacturing processes.

Typical manufacturing processes commonly used in production of various dosage forms can result in unwanted loss of one or more volatile lipophilic compounds in a final dosage form product at the end of the manufacturing process as compared to a starting amount of one or more lipophilic compounds added at the beginning of the manufacturing processes. Such typical manufacturing processes can greatly reduce the amount of volatile oil in the final product through the application of mixing, shear, granulation, blending, heating or cooking, pressure, among other processes, that may increase vapor pressure and/or increase airflow to the volatile lipophilic compounds.

The significant loss of concentration of volatile lipophilic compounds during manufacturing may impart a high cost of production, and a major source of variability of content within the finished dosage form.

There is a need in the art for new and improved methods for manufacturing various dosage forms containing one or more volatile lipophilic compounds that maintain concentrations of volatile lipophilic compounds during the manufacturing processes.

Accordingly, in some embodiments, the methods described herein provide mixtures, emulsions, compositions and/or dosage forms described herein that can minimize or prevent decrease of a starting amount and/or a starting concentration of the volatile lipophilic compounds during manufacture of the emulsions, compositions and/or dosage forms such that the amount or concentration of the volatile lipophilic compounds present in the emulsions, compositions and/or dosage forms is at least 70%, at least 75%, at least 80%, at least 85%, at least 90%, or at least 95% of the starting amount or the starting concentration of the volatile lipophilic compounds. Examples 19 and 20 of the present disclosure provide non-limiting example experimental results demonstrating increased concentrations of volatile lipophilic compounds using the methods described herein.

Without intending to be bound by theory, in some embodiments, the present disclosure relates to methods of providing mixtures and/or emulsions that are able to stabilize the one or more volatile lipophilic compounds in such a way that minimizes the volatility of the lipophilic compounds, such as by reducing vapor pressure during the manufacturing process. Without further intending to be bound by theory, vapor pressure of the one or more volatile lipophilic compounds may be reduced using the methods described herein by increasing solubility of the one or more volatile lipophilic compounds by the mixing and/or emulsifying steps described herein. Solubility of various volatile lipophilic compounds may be increased or decreased depending on the selection of formulation components and/or methods used to provide mixtures and/or emulsion.

The present disclosure relates to a method of providing a composition or a dosage form comprising one or more volatile lipophilic compounds. In some embodiments, the method comprises combining the one or more volatile lipophilic compounds with one or more water-insoluble components to provide a mixture. The method further comprises processing the mixture to provide the composition or the dosage form using one or more manufacturing methods capable of increasing vapor pressure of the one or more volatile lipophilic compounds when the one or more volatile lipophilic compounds are not combined with the one or more water-insoluble components. In other words, in absence of using the methods described herein, without combining the one or more volatile lipophilic compounds with one or more water-insoluble components to provide a mixture, the manufacturing methods used to provide the final dosage form product may result in increased vapor pressure of the one or more volatile lipophilic compounds during processing, resulting in decreased amount and/or concentration of the one or more volatile lipophilic compounds in the final dosage form product.

In some embodiments, the one or more manufacturing methods may include, without limitation, mixing, shearing, granulating, blending, heating or cooking, pressurizing, spray-drying, extrusion, spheronization, micro-encapsulation, and any combinations thereof.

In some embodiments, the present disclosure also relates to the mixtures, emulsions and/or dosage forms provided using the methods described herein.

The term vapor pressure refers to the pressure exerted by a vapor in thermodynamic equilibrium with its condensed phases (solid or liquid) at a given temperature. A substance or compound having a relatively high vapor pressure at a given temperature may be referred to as volatile. As temperature increases, the kinetic energy of a compound's molecules increases. As the kinetic energy of the molecules increases, the number of compounds transitioning into a vapor also increases, thereby increasing the vapor pressure. The vapor pressure of substances typically increases non-linearly with temperature according to the Clausius-Clapeyron relation.

Raoult's law gives an approximation to the vapor pressure of mixtures of liquids. Raoult's law states that the activity (e.g., pressure or fugacity) of a single-phase mixture is equal to the mole-fraction-weighted sum of the components' vapor pressures. Systems that have vapor pressures higher than would be expected according to Raoult's law are said to have positive deviations. Such a deviation suggests weaker intermolecular attraction than in the pure components, so that the molecules can be thought of as being retained the liquid phase less strongly than in the pure liquid. An example is the azeotrope of approximately 95% ethanol and water. Because the azeotrope's vapor pressure is higher than predicted by Raoult's law, it boils at a temperature below that of either pure component. There are also systems with negative deviations that have vapor pressures that are lower would be expected according to Raoult's law. Such a deviation is evidence for stronger intermolecular attraction between the constituents of the mixture than exists in the pure components. Thus, the molecules are retained in the liquid more strongly when a second molecule is present. An example is a mixture of trichloromethane (chloroform) and 2-propanone (acetone), which boils above the boiling point of either pure component.

Without intending to be limited to theory, vapor pressure of various volatile lipophilic compounds may be increased or decreased depending on the selection of formulation components and/or methods used to provide mixtures and/or emulsions. To the extent that formulation components can surround the volatile lipophilic compounds, the vapor pressure of the lipophilic compounds may be decreased. However, in some instances, if the formulation components that surround the volatile lipophilic compounds do not completely surround the volatile lipophilic compounds, the vapor pressure of the volatile lipophilic compounds may be unchanged. If the formulation components that are mixed with the volatile lipophilic compounds are easily stripped from the volatile lipophilic compounds, the vapor pressure may be increased when the volatile lipophilic compounds are subjected to various manufacturing processes described herein, such as mixing, drying, and high shear granulation, among others.

Vapor pressure of various substances, including but limited to one or more volatile lipophilic compounds, mixtures, and emulsions described herein, may be measured according to methods identifiable by skilled persons. For example, vapor pressure may be measured using methods known as the dynamic method, the equilibrium or static method, or the manometric method.

In some embodiments, the one or more volatile lipophilic compounds may have a first vapor pressure at a first temperature, the mixture may have a second vapor pressure at the first temperature, and the second vapor pressure may be lower than the first vapor pressure.

Accordingly, in some embodiments, without intending to be bound by theory, the present disclosure relates to methods of providing a stabilized emulsion comprising one or more volatile lipophilic compounds wherein the water-insoluble components may surround the volatile lipophilic compounds before processing into a dosage form using one or more dosage form manufacturing methods.

In some embodiments, the one or more water-insoluble components may be a component that is miscible with the one or more volatile lipophilic compounds. In some embodiments, the one or more water-insoluble components may comprise, without limitation, one or more oils, fats, waxes, polymers, and any combinations thereof.

In some embodiments, the one or more water-insoluble components may include, without limitation, coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether or other high molecular weight lipophilic materials, and any combinations thereof.

In some embodiments, the volatile lipophilic compounds can be one or more essential oils. The term “essential oils” as used herein refers to several classes of compounds that can be extracted from plants that capture the plants' scent and flavor, or “essence.” Essential oils may also be synthetic in origin. Particular combinations of compounds may impart an essential oil with its characteristic essence. The chemical composition of an essential oil may vary within the same plant species, or from plant to plant. Essential oils can be obtained through distillation (via steam and/or water) or mechanical methods, such as cold pressing. Once the chemicals have been extracted, they can be combined with a carrier oil.

In some embodiments, the essential oils can be one or more botanical essential oils. In some embodiments, botanical essential oils include, for example, without limitation, black pepper oils, thyme essential oil, clove oil, cinnamon oil, among other botanical essential oils identifiable by skilled persons.

Essential oils are perceived to provide a wide array of benefits to a human subject in need of relief. These uses include, but are not limited to, stress and anxiety reduction, headaches and migraines, sleep and insomnia, inflammation, and have the potential to provide antibiotic and antimicrobial benefits, as they do in the plants they are extracted from.

Essential oils can be widely used for their therapeutic benefits. They can be utilized in many types of aromatherapy, holistic therapy, and adjunctive therapy. For example, essential oils have been used as aromatherapy agents via topical application or burning natural materials that contain the oils. Aromatherapy uses include, but are not limited to, Cosmetic Aromatherapy, Massage Aromatherapy, Medical Aromatherapy, Olfactory Aromatherapy, and Psycho-aromatherapy. Essential oils can also be used in more traditional approaches due to their aroma, flavor, and potential therapeutic benefit.

Essential oils may also impart an entourage effect when administering cannabis or hemp-derived cannabinoids. The components of essential oils found in cannabis may include terpenes, terpenoids, and related classes of molecules. These essential oils can typically be administered through smoking and vaping materials for inhalation delivery, topically in cremes, balms, ointments, and patches, and ingested in oral dosage forms, among other methos of administration described herein.

Essential oils can typically be administered via three main routes of delivery: topical, inhaled, or oral ingestion. Accordingly, in some embodiments, the methods described herein can be used to provide stabilized emulsions that can be used in the manufacture of dosage forms for topical administration, inhalation administration, or oral ingestion, among others described herein.

Inhalation can be achieved by burning materials containing the essential oils within a room or an open container with volatile oil(s) so the vapor fills the room. In addition, the oils can be vaporized via smoking of the plant material in a cigarette or other device intended to vaporize the oils for a subject to inhale. These methods and devices include but are not limited to vaporizing and combustion, including devices to enable combustion or vaporization.

Topical administration of essential oils is performed using an appropriate dosage form or delivery device to provide the essential oils to the therapeutic site or for absorption into and through the skin. Devices such as patches have been shown to accurately and reproducibly deliver hydrophobic material across the skin for systemic delivery of drugs and medicaments. Semisolid dosage forms are used for topical delivery of drugs and medicaments. Examples of such semi-solid dosage forms include, but are not limited to, balms, gels, cremes, ointments, salves, and lotions.

Oral administration of essential oils can be achieved using a wide variety of dosage forms and delivery devices. This can be targeted at the sublingual cavity or buccal cavity or ingested to provide systemic exposure. There are a few approaches to delivery of drugs or medicaments via sublingual or buccal dosage forms, including semi-solid dosage forms, thin films, eroding or disintegrating tablets, mucoadhesive tablets, or an aerosolized spray. A spray could also be used for nasal instillation of the desired drugs or medicaments. Oral ingestion for systemic delivery can be achieved through multiple pathways, including but not limited to, tablets, capsules, granules, multiparticulates, sachets, lozenges, pills, modified-release tablets, medicated chews, elixirs, tinctures, and chewable tablets.

In some embodiments, the present disclosure relates to dosage forms including, but not limited to, solid or semi-solid dosage forms, such as compressed tablets, lozenges, troches, chewable tablets, mini-tablets, chocolates, confections, candies, soft chewable dosage forms such as gummies, gums, capsules, multi-particulate solids, such as powders, sprinkles, suspensions, bulk granulations, fast dissolving dosage forms such as fast dissolving tablets, fast-dissolving films, sublingual dosage forms such as tablets or films, buccal tablets or films. The present disclosure also relates to dosage forms including but not limited to semi-solid formulations such as creams, ointments, tinctures, sprays, suppositories, liniments, lotions, gels, pastes, balms, salves, infused bandages or patches, and inhaled dosage forms such aerosol inhalers, vape pens and devices, and aerosol forming devices, dry powder inhalers, nebulized formulations and powders or materials intended to be smoked and inhaled, either by combustion or electronic means. The dosage forms described herein may be used as medicinals, confectioneries, cosmetics, or food additives such as flavors or spices, or any combinations thereof.

The term “chewable dosage form” as used herein refers to an oral dosage form that can be chewed by a subject to cause release of one or more active ingredients from the dosage form, thereby allowing the active ingredients can be ingested orally by the subject. Chewable dosage forms offer several advantages. For example, chewable dosage forms are portable, stable, easy to manufacture, and they provide the accuracy and convenience when dosing. In various embodiments, volatile lipophilic compounds are useful components of flavor and aroma in chewable products.

Types of essential oils may include terpenes, terpenoids, and phenylpropanoids. Terpenes may further include types of compounds such as monoterpenes, sesquiterpenes, diterpenes, sesterterpenes, triterpenes, and tetraterpenes.

In some embodiments, the one or more essential oils may include, without limitation, myrcene, beta-caryophyllene, caryophyllene oxide, beta-eudesmol, limonene, linalool, alpha-pinene, beta-pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, cymene, octanol, isopulegol, cedrene, camphene, geranyl acetate, bergamotene, camphor, and pulegone.

Essential oils and a description of example benefits when administered to a subject may include, but are not limited to the following:

### Myrcene

Myrcene may contribute to the couch-lock effect of indica strains of cannabis, making high-myrcene hybrids useful as sleep aids or muscle relaxants.

### Beta-Caryophyllene

Beta-caryophyllene may act as an anti-inflammatory, promotes healthy digestion, and may enhance wound healing. Beta-caryophyllene also can bind directly to CB-2 receptors in the endocannabinoid system.

### Limonene

Limonene can be found in citrus plants and dill weed. Limonene affects several neurotransmitter pathways, which may make it helpful in treating depression and anxiety.

### Linalool

Scientists have been researching linalool's potential ability to reduce muscle spasms, relieve pain, and treat anxiety.

### Pinene

Pinene may reduce inflammation, aids memory, act as an antimicrobial agent, and open the respiratory passageways.

### Humulene

The medical cannabis community considers high-humulene strains useful for inflammation and weight control.

### Terpinolene

Terpinolene may help inhibit tumor growth and can have a positive effect on cardiovascular disease.

### Alpha-Bisabolol

Alpha-Bisabolol has shown potent antibacterial and antioxidant properties and may help reduce skin inflammation.

### Eucalyptol

Eucalyptol has potent antibacterial, antifungal, and insect-repelling properties. Eucalyptol may also help to lower blood pressure.

### Geraniol

Geraniol may have antimicrobial, antioxidant, antiviral, and neuroprotective properties. It may also be used as a mosquito repellent.

### Terpineol

Scientists are studying terpineol for its antioxidant, anti-anxiety, anti-malarial, and sedative effects.

### Farnesene

Farnesene may have neuroprotective properties and may help prevent certain types of cancer.

### Borneol

Borneol may impart pain-relieving and anti-inflammatory effects.

### Ocimene

Ocimene may have antioxidant, antimicrobial, and cancer-fighting properties.

### Nerolidol

Nerolidol has potent antifungal, antioxidant, antimicrobial, and anti-inflammatory properties.

### Guaiol

Guaiol may induce cell apoptosis in lung tumors.

### Valencene

Valencene may be used to repel insects, reduce inflammation, and fight skin cancer.

### Delta-3 Carene

Carene may help with neuropathic conditions like fibromyalgia and Alzheimer's disease and may help heal broken bones.

### Phytol

Phytol may be useful for pain-relief, reducing stress, and promoting healthy sleep patterns.

### Sabinene

Sabinene may help aid digestion, relieve arthritis, calm skin conditions, and prevent muscle atrophy.

### Phellandrene

Plants high in phellandrene may be useful for digestive problems, depression, and neuropathic pain.

### Fenchol

Fenchol can be found in fennel and is a common ingredient in perfumes.

### Menthol

Menthol may help kill bacteria and reduce inflammation.

### Terpinene

Terpinene may exhibit antitumor properties.

### Isoborneol

Isoborneol may have antiviral properties, particularly against herpes type 1. Additionally, Isoborneol may have antioxidant, anti-inflammatory, and antimicrobial abilities.

### Cymene

Cymene may have anti-inflammatory and pain-relieving properties.

In some embodiments, the additional essential oils may include, without limitation, one or more of Octanol, Isopulegol, Cedrene, Camphene, Geranyl Acetate, Bergamotene, Camphor, and Pulegon.

In some embodiments, after providing the mixture and before the dosage form manufacturing processing step or steps, the method may further comprise adding one or more surface-active agents and one or more water-soluble components to the mixture, and emulsifying the mixture comprising the one or more surface-active agents and the one or more water-soluble components to provide an emulsion. The emulsion may then be processed into a dosage form using one or more dosage form manufacturing methods.

The terms “surfactant,” “emulsifier,” or “surface-active agent,” as used herein refers in general to a substance which tends to reduce the surface tension of a liquid in which it is dissolved, thereby increasing its spreading and wetting properties.

Surfactants are widely used across many industries and applications, including, but not limited to, detergents, fabric softeners, emulsifying agents in emulsions, paints, adhesives, inks, anti-fog formulations, suspending agents in suspensions, laxatives, firefighting, pipelines, alkali surfactant polymers (used to mobilize oil in oil wells), ferrofluids, and leak detectors.

There are 4 main descriptive categories of surfactants, including anionic, cationic, neutral, and amphoteric or zwitterionic. These categories are defined by the ionization state(s) of the molecule.

Anionic surfactants contain permanent anions or pH-dependent anions and can interact with a substrate based on this ionization state. Commonly used anionic surfactant are based on aliphatic carboxylic acids, which can be derived from naturally occurring animal and plant fats. The carboxylic acids are retrieved as their sodium or potassium salts, which makes them anionic surfactants. Because they can be obtained from fats, these aliphatic carboxylic acids are often referred to as fatty acids.

Cationic surfactants are based on pH-dependent primary, secondary or tertiary amines, and have a positive charge on the hydrophilic end of the molecule.

Nonionic surfactants do not include any charge head at any pH value. Nonionics have no charge on their hydrophilic end, which helps make them superior oily soil emulsifiers.

Amphoteric or zwitterionic surfactants can carry a positive charge on a cationic site and a negative charge on an anionic site. Amphoteric surfactants can serve as coupling agents, which hold the surfactants, solvents and inorganic salt components of a formula together.

In some embodiments, suitable surfactants may be included at 0.1%-5%, such as at, or about at, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0%. Suitable surfactants may include but are not limited to, polysorbates, such as for example, polyoxyethylene (POE)-20-sorbitan monooleate, Tween 80, Crillet 4, polyoxyethylene (POE)-20-sorbitan monolaurate. Tween 60, Tween 40, Tween 20, and Crillet 1; sorbitan esters, such as for example, sorbitan monolaurate, Span 20, and Crill 1; ((Poly(ethylene oxide))-(poly(propylene oxide)) (PEO-PPO)-block copolymers, such as for example, Poloxamer 188, Pluronic/Lutrol F 68; polyoxyethylene (POE) alkyl ethers, such as for example, polyoxyethylene (POE)-10-oleyl ether and Brij 96 V; polyoxyethylene (POE) castor oil, such as for example, polyoxyethylene (POE)-35-castor oil, Cremophor-L (also named Kolliphor EL), and Etocas 35 HV; polyoxyethylene (POE) hydrogenated castor oil, such as for example, polyoxyethylene (POE)-40-hydrogenated castor oil, Cremophore RH 40 (also named Kolliphor RH 40), hydrogenated castor oil (HCO)-40, and Croduret 40 LD; polyoxyethylene (POE)-60-hydrogenated castor oil, such as for example, Cremophore RH 60, hydrogenated castor oil (HCO)-60; polyoxyethylene (POE)-stearate, such as for example, polyethyleneglycol (PEG)-660-12-hydroxystearate and Solutol HS 15 (also named Kolliphor HS15); phospholipids, such as for example, soybean lecithin, egg lecithin, diolelyl phosphatidyl choline, and distearoyl phosphatidyl glycerol; polyethyleneglycol (PEG)ylated phospholipids; dimyristoyl phosphatidyl choline; Span 80; and Brij 97; or mixtures thereof.

In some embodiments, the one or more surface-active agents may include, without limitation, glyceryl monostearate, lecithin, a Tween, a Span, a modified food starch, a monoglyceride, a diglyceride, a cellulose derivative, guar gum, carrageenan, Acacia gum, a hydrocolloid, a fatty ester, a long-chain fatty acid, sodium citrate, pectin, and any combinations thereof.

In some embodiments, the one or more volatile lipophilic compounds may have a first vapor pressure at a first temperature, the emulsion may have a third vapor pressure at the first temperature; and the third vapor pressure may be lower than the first vapor pressure.

In some embodiments, the method further comprises processing the emulsion to provide the dosage form using one or more manufacturing methods capable of increasing vapor pressure of the one or more volatile lipophilic compounds when the one or more volatile lipophilic compounds are not provided as an emulsion of the present disclosure. In other words, in absence of using the methods described herein, without providing the one or more volatile lipophilic compounds as an emulsion of the present disclosure, the manufacturing methods used to provide the final dosage form product may result in increased vapor pressure of the one or more volatile lipophilic compounds during processing, resulting in decreased amount and/or concentration of the one or more volatile lipophilic compounds in the final dosage form product.

In some embodiments, the one or more water-soluble components may include, without limitation, gelatin, whey, casein, albumin, pectin, modified food starch, microcrystalline cellulose, hydroxypropyl methylcellulose, hydroxypropyl cellulose, polyvinyl alcohol, polyvinylpolypyrrolidone, beta celluloses, and any combinations thereof.

In some embodiments, the mixing may be performed using a high-shear mixer. In some embodiments, the emulsifying may be performed using a high-shear mixer.

Accordingly, in some embodiments, the present disclosure relates to methods of providing a stabilized emulsion comprising one or more volatile lipophilic compounds.

In some embodiments, the one or more surface-active agents may comprise a proteinaceous material. In some embodiments, the one or more proteinaceous materials may include, without limitation, albumin, gelatin, whey protein, a caseinate, a plant-based protein, and any combinations thereof.

The dosage forms described herein may further comprise one or more active pharmaceutical ingredients.

Other materials that can be used in the methods described herein, besides the active ingredients, volatile lipophilic compounds, water soluble or water insoluble components, and surface-active agents include but are not limited to other proteinaceous materials, fillers and/or binders, and other materials such as flavors, colors, and minor components. These materials can be water soluble or water insoluble.

In some embodiments, the proteinaceous materials may include, without limitation, gelatin that can be used at from 3% to 10% (e.g., about 3%, 4%, 5%, 6%, 7%, 8%, 9%, or 10%) of the final formula; egg albumin that can be used at from 1% to 5% (e.g., about 1%, 2%, 3%, 4%, or 5%) of the final formula; dairy proteins that can be used at from 0.5% to 2% (e.g., about 0.5%, 1%, 1.5%, or 2%) of the final formula; and plant proteins (e.g., soy) that can be used at from 0.5% to 2% (e.g., about 0.5%, 1%, 1.5%, or 2%) of the final formula.

In some embodiments, fillers/binders materials may include, without limitation, mono- and di-saccharides, including but not limited to sucrose, maltose, glucose, and fructose that can be used at from 30% to 65% (e.g., about 30%, 35%, 40%, 45%, 50%, 55%, 60%, or 65%) of the final formula; glucose syrups, including, but not limited to corn, rice, and tapioca syrups that can be used at from 30% to 65% (e.g., about 30%, 35%, 40%, 45%, 50%, 55%, 60%, or 65%) of the final formula; pectin, both low methoxy and high methoxy pectins that can be used at from 0.5% to 2% (e.g., about 0.5%, 1%, 1.5%, or 2%) of the final formula; gum Arabic that can be used at from 15% to 45% (e.g., about 15%, 20%, 25%, 30%, 35%, 40%, or 45%) of the final formula; and agar that can be used at from 1% to 2% (e.g., about 1%, 1.5%, or 2%) of the final formula.

In some embodiments, other materials may include, without limitation, artificial, natural, nature identical, and natural with other natural flavors water soluble flavors that can be used at from 0.01% to 5% of the final formula; artificial, natural, nature identical, and natural with other natural flavors oil soluble flavors that can be used at from 0.01% to 5% of the final formula; maillard or other process flavor systems developed through thermal, and or chemical reactions in-situ; flavor and aroma modifying products, e.g., sugars, sugar alcohols, proteins, lipids, and metal salts of the above that can be used at from 0.01% to 3% (e.g., about 0.01%, 0.5%, 1%, 1.5%, 2%, 2.5%, or 3%) of the final formula; natural colors derived from plants, microbiological and animals; artificial colors; and minor components of flavors include but not limited to: emulsifying systems to develop water miscible flavors or colors from lipophilic starting materials; emulsifying systems to develop oil miscible flavors or colors from lipophobic/hydrophilic starting materials; anti-oxidants and of chemical stabilizing systems for flavor and coloring systems that can be to prevent off-flavor or color degradation.

In some embodiments, after providing the emulsion and before the dosage form manufacturing processing step or steps, the method may further comprise at least partially covering the emulsion with a layer of one or more water-insoluble components to provide a covered emulsion. The one or more water-insoluble components may comprise one or more oils, fats, waxes, polymers, or any combinations thereof. The one or more water-insoluble components may include, without limitation, coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether, and any combinations thereof.

In some embodiments, the one or more volatile lipophilic compounds may have a first vapor pressure at a first temperature, the covered emulsion may have a fourth vapor pressure at the first temperature; and the fourth vapor pressure may be lower than the first vapor pressure.

In some embodiments, the method further comprises processing the covered emulsion to provide the dosage form using one or more manufacturing methods capable of increasing vapor pressure of the one or more volatile lipophilic compounds when the one or more volatile lipophilic compounds are not provided as a covered emulsion of the present disclosure. In other words, in absence of using the methods described herein, without providing the one or more volatile lipophilic compounds as a covered emulsion of the present disclosure, the manufacturing methods used to provide the final dosage form product may result in increased vapor pressure of the one or more volatile lipophilic compounds during processing, resulting in decreased amount and/or concentration of the one or more volatile lipophilic compounds in the final dosage form product.

In some embodiments, the method may further comprise combining a mixture or an emulsion or covered emulsion described herein with one or more additional components and processing using one or more manufacturing methods to provide a chewable dosage form. For example, suitable additional components may include, without limitation, gelatin, xanthan gum, guar gum, pectin, modified starch, and any combinations thereof.

In some embodiments, the chewable dosage mixture may be converted into a chewable dosage form by a manufacturing method comprising tableting, molding, rolling, depositing, slabbing, or any combinations thereof.

Non-limiting examples of manufacturing chewable dosage forms of the present disclosure are described below.

### Tableted Chews

A pharmaceutical tablet containing volatile lipophilic compounds with or without another active ingredient can be manufactured by any of the method such as direct compression, dry granulation or wet granulation or fluidized technique or multi-particulates using an extrusion spheronization process.

One method for manufacturing an immediate release tablet containing volatile lipophilic compounds, with or without another active ingredient, a binder, a diluent, a disintegrant, a glidant, flavor(s), and a lubricant comprises: a) adding the binder and a first portion of diluent to volatile lipophilic compounds and then adding a second portion of the diluent and flavors to form a first mixture; b) mixing the first mixture; c) a third portion of the diluent, the disintegrant, and the glidant to form a second mixture; d) adding a first portion of the second mixture to the first mixture and then adding the remaining portion of the second mixture to form a third mixture; e) mixing the third mixture; f) lubricating the third mixture by addition of a lubricant; g) compressing the lubricated mixture into a tablet; and h) optionally coating the tablet, wherein the coating is a functional or non-functional coating.

Another method for manufacturing an immediate release tablet containing volatile lipophilic compounds with or without another active ingredient, a binder, a diluent, a disintegrant, flavor(s), a glidant, and a lubricant comprises: a) sifting all the ingredients including the volatile lipophilic compounds with or without another active ingredient, binder, diluent, disintegrant, glidant, and lubricant; b) dividing the diluent into two equal parts, part 1 and part 2; c) adding approximately half of part 1 of the diluent and the volatile lipophilic compounds to a suitable blender and then adding the remaining half of part 1 of the diluent to the blender; d) mixing the step c) blend (for example, for 10 minutes at 15 rpm (such as 150 tumbles) to make a premix of volatile lipophilic compounds; e) sifting the step d) blend through a 40 #sieve; f) co-sifting part 2 of the binder, diluent, the disintegrant, and the glidant through 40 #sieve; g) adding half of the step f) blend and the step e) blend to a suitable blender and then adding the remaining half of the step f) blend; h) mixing of the step g) blend in a suitable blender (for example, for 30 minutes at 15 rpm (such as 450 tumbles)); i) lubricating the step h) blend with a lubricant; j) compressing the step i) blend to form a tablet (for instance, using suitable punches such as round/capsule/oval shaped punches and optionally with score line for dose adjustment); and k) optionally coating the tablet wherein the coating is a functional or non-functional coating.

Yet another method for manufacturing an immediate release tablet containing volatile lipophilic compounds with or without another active ingredient comprises: a) dissolving the volatile lipophilic compounds and flavor(s) with or without another active ingredient in ethanol to form a solution; b) granulating the binder and diluent with the ethanol solution of step a); c) drying the granulation product of step b) with an optional classification of the granules; d) blending the granulation with additional diluent, disintegrant, and glidant; e) lubricating the blend from step d); f) compressing the mixture of step e) into a tablet (e.g., using round/capsule/oval shaped punches and optionally with a score line for dose adjustment); and g) optionally coating the tablet wherein the coating comprises a functional or non-functional coating.

Yet another method for manufacturing an immediate release tablet containing volatile lipophilic compounds with or without another active ingredient, hypromellose, microcrystalline cellulose, croscarmellose sodium, silicon dioxide, and magnesium stearate comprising: a) sifting of volatile lipophilic compounds, hypromellose, microcrystalline cellulose, croscarmellose sodium, silicon dioxide, and magnesium stearate; b) dividing microcrystalline cellulose into two equal parts, part 1 and part 2; c) adding approximately half of the part 1 hypromellose/microcrystalline cellulose to the volatile lipophilic compounds with or without another active ingredient and then adding the remaining half of the part 1 hypromellose/microcrystalline cellulose to a suitable blender; d) mixing the step c) blend (for example, for 10 minutes at 15 rpm (e.g., for 150 tumbles); e) sifting the step d) blend through a 40 #sieve; f) co-sifting of the part 2 hypromellose/microcrystalline cellulose, croscarmellose sodium and silicon dioxide through a 40 #sieve; g) adding half of the step f) blend to the sifted step e) blend and then adding the remaining half of the step f) blend to a suitable blender; h) mixing of the step g) blend in a suitable blender (e.g., for 30 minutes at 15 rpm (e.g., for 450 tumbles)); i) lubricating the step h) blend with magnesium stearate; j) compressing the lubricated blend to form a tablet (for example, using suitable punches such as round/capsule/oval shaped punches and optionally with a score line for dose adjustment); and k) optionally coating the tablet wherein the coating comprises a functional or non-functional coating.

### Molded Chews

A pharmaceutical chew or tablet containing volatile lipophilic compounds with or without another active ingredient can be manufactured by any of the depositing methods such as starch molding, silicon molding, or molding into polycarbonate molds.

One method for manufacturing a molded chew containing volatile lipophilic compounds, with or without another active ingredient, water, a hydrocolloid, a sweetening agent, a bulking agent, a buffering agent, an acid, a color(s), a flavor(s), and a hydrophobic lubricant comprises: a) adding the flavor and other active ingredient to volatile lipophilic compounds and then adding the water, a buffering agent, and a hydrocolloid to form a first mixture; b) mixing the first mixture; c) adding a sweetening agent and a bulking agent to form a second mixture; d) adding the first mixture to the second mixture to form a third mixture; e) cooking the third mixture to 78-80% soluble solids; f) adding color and acid to reach a final pH of 3.2-3.6 and mixing to form the fourth mixture; h) depositing the cooked mixture using a heated depositor into the desired mold; and i) lubricating the resultant chew upon demolding.

Another method for manufacturing a molded chew containing volatile lipophilic compounds, with or without another active ingredient, water, a hydrocolloid, a sweetening agent, a bulking agent, a buffering agent, an acid, a color(s), a flavor(s), and a hydrophobic lubricant comprises: a) adding the other active ingredient to volatile lipophilic compounds and then adding the water, a buffering agent, and a hydrocolloid to form a first mixture; b) mixing the first mixture; c) adding a sweetening agent and a bulking agent to form a second mixture; d) adding the first mixture to the second mixture to form a third mixture; e) cooking the third mixture to 78-80% soluble solids; f) adding color(s), flavor(s) and acid to reach a final pH of 3.2-3.6 and mixing to form the fourth mixture; h) depositing the cooked mixture using a heated depositor into the desired mold; and i) lubricating the resultant chew upon demolding.

Yet another method for manufacturing a molded chew containing volatile lipophilic compounds, with or without another active ingredient, water, a protein, a sweetening agent, a bulking agent, a buffering agent, an acid, a color(s), a flavor(s) and a hydrophobic lubricant comprises: a) adding active ingredient(s) and a portion of the flavor to volatile lipophilic compounds, and then adding a buffer and a portion of the water to form a first mixture; b) mixing the first mixture; c) adding sweetening agent and bulking agent to form a second mixture; d) adding the remainder of the water to the protein to solubilize the protein and form a third mixture; e) adding the first mixture to the second mixture to form a fourth mixture; f) cooking the fourth mixture to 80-82% soluble solids; g) add the third mixture to the fourth mixture to form a fifth mixture; e) add color(s), the remaining flavor(s), and acid and mixing to form the sixth mixture; h) depositing the mixture into the desired mold; and i) lubricating the resultant chew upon demolding.

Yet another method for manufacturing a molded chew containing volatile lipophilic compounds, with or without another active ingredient, water, a hydrocolloid, a sweetening agent, a bulking agent, an acid, a color(s), a flavor(s), and a hydrophobic lubricant comprises: a) adding the other active ingredient to volatile lipophilic compounds and then adding the water and a hydrocolloid to form a first mixture; b) mixing the first mixture; c) adding a sweetening agent and a bulking agent to form a second mixture; d) adding the first mixture to the second mixture to form a third mixture; e) cooking the third mixture to 78-80% soluble solids; f) adding color(s), flavor(s) and acid to reach a final pH of 3.2-3.6 and mixing to form the fourth mixture; h) depositing the cooked mixture using a heated depositor into the desired mold; and i) lubricating the resultant chew upon demolding.

### Rolled Chews

One method for manufacturing a rolled chew containing volatile lipophilic compounds, with or without another active ingredient, water, an emulsifier(s), a sweetening agent, a bulking agent, a hydrophobic saturated lubricant, a protein, an acid, a color(s), and a flavor(s), comprises: a) melting the lubricant and adding the emulsifier(s), active ingredient(s), and volatile lipophilic compounds to form a first mixture; b) mixing the first mixture; c) adding a portion of the water to the protein to hydrate the protein and form the second mixture; d) adding the sweetening agent, bulking agent, and the remainder of the water to form a third mixture; d) adding the first mixture to the third mixture to form a fourth mixture; e) cooking the fourth mixture to 80-82% soluble solids; f) cooling the cooked mass to 65° C.; g) adding the second mixture to the fourth mixture to form a fifth mixture; h) discharging the mixed mass onto a cooling table, and mixing to incorporate the color(s), flavor(s) and acid; g) placing the resultant mass on forming rolls, and feeding the rolled mass through appropriate cut and wrap equipment.

Another method for manufacturing a rolled chew containing volatile lipophilic compounds, with or without another active ingredient, water, an emulsifier(s), a sweetening agent, a bulking agent, a hydrophobic saturated lubricant, a modified starch, an acid, a color(s), and a flavor(s), comprises: a) adding the starch(es), a portion of the water, the emulsifier(s), and the lubricant to form a first mixture; b) mix the first mixture; c) adding the sweetening agent, the remainder of the water, the bulking agent, and the color to the first mixture to form the second mixture; d) cooking the second mixture to 82-83% soluble solids; d) creating a third mixture by adding the flavor(s), volatile lipophilic compounds, and active ingredient(s) to form a third mixture; e) adding the third mixture to the cooked second mixture to form a fourth mixture; f) kneading the fourth mixture with a Z blender to fully emulsify the mixture; h) discharging the mixed mass onto a cooling table, and mixing to incorporate the acid; g) placing the resultant mass on forming rolls, and feeding the rolled mass through appropriate cut and wrap equipment.

### Others

One method for manufacturing an deposited chew containing volatile lipophilic compounds, with or without another active ingredient, water, a hydrocolloid, a sweetening agent, a bulking agent, a buffering agent, an acid, a color(s), a flavor(s), and a hydrophobic lubricant comprises: a) adding the flavor and other active ingredient to volatile lipophilic compounds and then adding the water, a buffering agent, and a hydrocolloid to form a first mixture; b) mixing the first mixture; c) adding a sweetening agent and a bulking agent to form a second mixture; d) adding the first mixture to the second mixture to form a third mixture; e) cooking the third mixture to 78-80% soluble solids; f) adding color and acid to reach a final pH of 3.2-3.6 and mixing to form the fourth mixture; h) depositing the cooked mixture using a heated depositor into the desired mold; and i) lubricating the resultant chew upon demolding.

Another method for manufacturing an deposited or slabbed chew containing volatile lipophilic compounds, with or without another active ingredient, water, a hydrocolloid, a sweetening agent, a bulking agent, a buffering agent, an acid, a color(s), a flavor(s), and a hydrophobic lubricant comprises: a) adding the flavor and other active ingredient to volatile lipophilic compounds and then adding the water, a buffering agent, and a hydrocolloid to form a first mixture; b) mixing the first mixture; c) adding a sweetening agent and a bulking agent to form a second mixture; d) adding the first mixture to the second mixture to form a third mixture; e) cooking the third mixture to 78-80% soluble solids; f) adding color and acid to reach a final pH of 3.2-3.6 and mixing to form the fourth mixture; h) pouring the cooked mixture on a lubricated surface or pan and allowing the product to cool; i) cutting the chew to the desired shape and size for proper dosage; and f) lubricating the resultant chew after cutting.

Yet another method for manufacturing an deposited chew containing volatile lipophilic compounds, with or without another active ingredient, a saturated fat, a nutritive or non-nutritive sweetener, a bulking agent, an emulsifier, a color(s), and a flavor(s), comprises: a) melting the saturated fat and adding the sweetener and bulking agent; b) applying grinding pressure to the resultant mass to reduce the particle size of the sweetener and bulking agent to <30 μm to form the first mixture; c) mixing the volatile lipophilic compounds, an emulsifier, and a portion of the flavor(s) to form a second mixture; d) adding the second mixture to the first mixture to firm a third mixture; e) applying an appropriate heating and cooling regimen to the third mixture to temper the fatty acids in the saturated fat mass; f) adding the color and additional flavor to the third mixture to form a fourth mixture; g) pouring the mixture on a lubricated surface or pan and allowing the product to cool; h) cutting the chew to the desired shape and size for proper dosage.

Yet another method for manufacturing an molded chew containing volatile lipophilic compounds, with or without another active ingredient, water, a nutritive or non-nutritive sweetener, a hydrocolloid(s), an protein, a color(s), and a flavor(s), comprises: a) mixing the hydrocolloid(s) with a portion of the water to hydrate fully to form the first mixture; b) mixing the protein with a portion of the water to hydrate the protein and form the second mixture; c) mixing the volatile lipophilic compounds, active ingredient, and a portion of the flavor(s) to form a third mixture; d) add the third mixture to the sweetener and blend to form a fifth mixture; e) add the first and second mixtures to the fifth mixture and mix slowly and thoroughly to form a sixth mixture; e) discharge the mixer and put the sixth mixture into a feed hopper; i) stamp out the final product; and j) stove the chews at 35-40° C./<30% relative humidity for 24 hours prior to final packaging.

In some embodiments, the present disclosure provides compositions and dosage forms comprising one or more volatile lipophilic compounds. In some embodiments, the one or more volatile lipophilic compounds are present in the composition in an amount from 0.001% to 15% by weight of the composition. In some embodiments, the one or more volatile lipophilic compounds are present in the dosage form in an amount from 0.001% to 15% by weight of the dosage form.

In some embodiments, one or more volatile lipophilic compounds are present in the composition or the dosage form in an amount (w/w %) of at least: 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0.

In some embodiments, one or more volatile lipophilic compounds are present in the composition or the dosage form in an amount (w/w %) of up to: 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0.

In some embodiments, the total amount of a plurality of volatile lipophilic compounds are present in the composition or the dosage form in an amount (w/w %) of at least: 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0.

In some embodiments, the total amount of a plurality of volatile lipophilic compounds are present in the composition or the dosage form in an amount (w/w %) of up to: 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0.

In some embodiments, the amount of one or more volatile lipophilic compounds that are present in a composition or in a dosage form may be determined using any suitable quantitative method known in the art, For example, without limitation, the amount of one or more volatile lipophilic compounds that are present in a composition or in a dosage form may be determined using mass spectrometry, for example such as in Example 20.

In some embodiments, the present disclosure provides compositions and dosage forms comprising a mixture of one or more volatile lipophilic compounds and one or more water-insoluble components, wherein the one or more volatile lipophilic compounds are present in an amount from 0.001% to 15% by weight of the composition or the dosage form.

In some embodiments, the one or more volatile lipophilic compounds may have a first vapor pressure at a first temperature; the mixture may have a second vapor pressure at the first temperature; and the second vapor pressure may be lower than the first vapor pressure.

In some embodiments, the composition may comprise an emulsion of the mixture and one or more surface-active agents and one or more water-soluble components. In some embodiments, the emulsion may have a third vapor pressure at the first temperature, and the third vapor pressure may be lower than the first vapor pressure.

In some embodiments, the one or more volatile lipophilic compounds in the composition or the dosage form may comprise one or more essential oils. For example, and not by way of limitation, the one or more essential oils may be selected from the group consisting of: myrcene, beta-caryophyllene, caryophyllene oxide, beta-eudesmol, limonene, linalool, alpha-pinene, beta-pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, cymene, octanol, isopulegol, cedrene, camphene, geranyl acetate, bergamotene, camphor, pulegone, and any combinations thereof.

The one or more water-insoluble components may comprise one or more oils, fats, waxes, polymers, or any combinations thereof. For example, and not by way of limitation, the one or more water-insoluble components may be selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether, and any combinations thereof.

For example, and not by way of limitation, the one or more surface-active agents may be selected from the group consisting of: glyceryl monostearate, lecithin, a Tween, a Span, a modified food starch, a monoglyceride, a diglyceride, a cellulose derivative, guar gum, carrageenan, Acacia gum, a hydrocolloid, a fatty ester, a long-chain fatty acid, sodium citrate, pectin, and any combinations thereof.

For example, and not by way of limitation, the one or more water-soluble components may be selected from the group consisting of: gelatin, whey, casein, albumin, pectin, modified food starch, microcrystalline cellulose, hydroxypropyl methylcellulose, hydroxypropyl cellulose, polyvinyl alcohol, polyvinylpolypyrrolidone, beta celluloses, and any combinations thereof.

In some embodiments, the composition may be formulated as a dosage form.

For example, and not by way of limitation, the dosage form may be selected from the group consisting of: a solid dosage form, a soft chewable dosage form, a multi-particulate solid, a fast dissolving dosage form, a semi-solid dosage form, and an inhaled dosage form.

For example, and not by way of limitation, the solid dosage form may be a compressed tablet, a lozenge, a troche, a chewable tablet, a capsule, a mini-tablet, or any combinations thereof.

For example, and not by way of limitation, the soft chewable dosage form may be a gummy, a gum, or a combination thereof.

The gum may comprise one or more gum-forming components. For example, and not by way of limitation, gum-forming components may be selected from the group consisting of: gelatin, xanthan gum, guar gum, pectin, modified starch, and any combinations thereof.

For example, and not by way of limitation, the multi-particulate solid may be a powder, a sprinkle, a suspension, a bulk granulation, or any combinations thereof.

For example, and not by way of limitation, the fast dissolving dosage form may be a fast dissolving tablet, a fast-dissolving film, a sublingual tablet, a sublingual film, a buccal tablet, a buccal film, or any combinations thereof.

For example, and not by way of limitation, the semi-solid dosage form may be a cream, an ointment, a tincture, a spray, a suppository, a liniment, a lotion, a gel, a paste, a balm, a salve, an infused bandage, an infused patch, or any combinations thereof.

For example, and not by way of limitation, the inhaled dosage form may be a dosage form administrable with an aerosol inhaler, a vape pen, a vape device, and aerosol forming device, a dry powder inhaler, a nebulizer, or any combinations thereof.

For example, and not by way of limitation, the inhaled dosage form may be administrable by smoking and inhaling, wherein the smoking may be by combustion, electronic means, or a combination thereof.

In some embodiments, the composition may further comprise a layer of one or more water-insoluble components disposed around the emulsion.

For example, and not by way of limitation, the one or more water-insoluble components may comprise one or more oils, fats, waxes, polymers, or any combinations thereof.

For example, and not by way of limitation, the one or more water-insoluble components may be selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether and any combinations thereof.

In some embodiments, the one or more surface-active agents may comprise one or more proteinaceous materials.

For example, and not by way of limitation, the one or more proteinaceous materials may be selected from the group consisting of: albumin, gelatin, whey protein, a caseinate, a plant-based protein, and any combinations thereof.

For example, and not by way of limitation, Examples 1-20 of the present disclosure provide example formulations of example chewable dosage forms containing volatile lipophilic compounds (e.g., terpenes), and analytical results reporting example levels of volatile lipophilic compounds (e.g., terpenes) present in the example chewable dosage forms.

For example, in some embodiments, the present disclosure provides compositions and dosage forms having higher levels of volatile lipophilic compounds (e.g., terpenes) than may be possible without using the methods described herein. For example, and not by way of limitation, Examples 19 and 20 of the present disclosure present example analytical results of terpene content in example chewable dosage forms produced according to the present disclosure. Example 20 also presents example analytical results of terpene content in example chewable dosage forms produced by other manufacturers, without using the methods according to the present disclosure. As shown for example in Example 20, the example chewable dosage forms manufactured according to the methods described herein, have much higher terpene content than the example chewable dosage forms from other manufacturers. In the example dosage forms from other manufacturers, most terpenes were not detectable, and the terpenes that were detectable were only present in very low levels. These results provide further evidence to support the superior utility of the methods described herein for maintaining concentrations of volatile lipophilic compounds in manufacture of dosage forms.

In some embodiments, for example and not by way of limitation, as shown in Example 20, the compositions and dosage forms of the present disclosure may have a terpene content in an amount from 0.001% to 15% by weight of the composition or by weight of the dosage form. In some embodiments, for example, and not by way of limitation, the terpenes may include one or more of myrcene, limonene, alpha-pinene, beta-pinene, beta-caryophyllene, linalool, and ocimene, in any combination.

### EXAMPLES

The present examples are provided for illustrative purposes only. They are not intended to and should not be interpreted to encompass the full breadth of the disclosure.

### Examples 1-18. Example Formulations of Chewable Dosage Forms Containing Volatile Lipophilic Compounds

The percentages referred to in the following examples refers to Ingredient Conc. (% W/W).

### Example 1

| Sucrose | 60% w/w |
|---|---|
| Rice Syrup | 10% |
| Low-methoxy pectin | 1.0% |
| Gum Arabic | 14% |
| Gelatin | 2.0% |
| Milk proteins | 1.0% |
| Linalool | 4% |
| Limonene | 4% |
| β-Caryophyllene | 4% |
| Total | 100% |

### Example 2

| Sucrose | 60% w/w |
|---|---|
| Rice Syrup | 10% |
| Low-methoxy pectin | 1.0% |
| Sodium citrate | 4% |
| Gum Arabic | 10% |
| Gelatin | 2.0% |
| Milk proteins | 1.0% |
| Linalool | 4% |
| Limonene | 4% |
| β-Caryophyllene | 4% |
| Total | 100% |

### Example 3

| Glucose | 30% w/w |
|---|---|
| Maltose | 30% |
| Rice Syrup | 10% |
| Low-methoxy pectin | 1.0% |
| Glyceryl monostearate | 14% |
| Gelatin | 2.0% |
| Milk proteins | 1.0% |
| Linalool | 4% |
| Limonene | 4% |
| β-Caryophyllene | 4% |
| Total | 100% |

### Example 4

| Sucrose | 50% w/w |
|---|---|
| Maltose | 20% |
| Low-methoxy pectin | 1.0% |
| Corn syrup | 10% |
| Gelatin | 4.0% |
| Lecithin | 2.0% |
| Milk proteins | 1.0% |
| Linalool | 4% |
| Limonene | 4% |
| β-Caryophyllene | 4% |
| Total | 100% |

### Example 5

| Sucrose | 50% w/w |
|---|---|
| Maltose | 20% |
| Low-methoxy pectin | 1.0% |
| Corn syrup | 10% |
| Gelatin | 4.0% |
| Tween 20 | 2.0% |
| Milk proteins | 1.0% |
| Limonene | 8% |
| β-caryophyllene | 4% |
| Total | 100% |

### Example 6

| Sucrose | 50% w/w |
|---|---|
| Maltose | 20% |
| Low-methoxy pectin | 1.0% |
| Corn syrup | 10% |
| Gelatin | 4.0% |
| Span 80 | 2.0% |
| Milk proteins | 1.0% |
| Limonene | 8% |
| β-caryophyllene | 4% |
| Total | 100% |

### Example 7

| Sucrose | 50% w/w |
|---|---|
| Maltose | 20% |
| Low-methoxy pectin | 1.0% |
| Corn syrup | 10% |
| Gelatin | 4.0% |
| Pregelatinized starch | 2.0% |
| Milk proteins | 1.0% |
| Limonene | 8% |
| β-caryophyllene | 4% |
| Total | 100% |

### Example 8

| Sucrose | 50% w/w |
|---|---|
| Maltose | 20% |
| Low-methoxy pectin | 1.0% |
| Corn syrup | 10% |
| Gelatin | 4.0% |
| Soybean oil | 2.0% |
| Milk proteins | 1.0% |
| Limonene | 8% |
| β-caryophyllene | 4% |
| Total | 100% |

### Example 9

| Sucrose | 50% w/w |
|---|---|
| Maltose | 20% |
| Low-methoxy pectin | 1.0% |
| Corn syrup | 10% |
| Gelatin | 4.0% |
| Cottonseed oil | 2.0% |
| Milk proteins | 1.0% |
| Limonene | 8% |
| β-caryophyllene | 4% |
| Total | 100% |

### Example 10

| Sucrose | 50% w/w |
|---|---|
| Maltose | 20% |
| Low-methoxy pectin | 1.0% |
| HPMC-Hydroxypropyl Methylcellulose | 10% |
| Gelatin | 4.0% |
| Soybean oil | 2.0% |
| Milk proteins | 1.0% |
| Limonene | 8% |
| β-caryophyllene | 4% |
| Total | 100% |

### Example 11

| Sucrose | 50% w/w |
|---|---|
| Maltose | 20% |
| Low-methoxy pectin | 10% |
| Guar Gum | 10% |
| Gelatin | 4.0% |
| Soybean oil | 2.0% |
| Milk proteins | 1.0% |
| Limonene | 8% |
| β-caryophyllene | 4% |
| Total | 100% |

### Example 12

| Sucrose | 50% w/w |
|---|---|
| Maltose | 20% |
| Low-methoxy pectin | 1.0% |
| Carrageenan | 10% |
| Gelatin | 4.0% |
| Soybean oil | 2.0% |
| Milk proteins | 1.0% |
| Limonene | 8% |
| β-caryophyllene | 4% |
| Total | 100% |

### Example 13

| Sucrose | 50% w/w |
|---|---|
| Maltose | 20% |
| Low-methoxy pectin | 1.0% |
| Acacia gum | 10% |
| Gelatin | 4.0% |
| Soybean oil | 2.0% |
| Milk proteins | 1.0% |
| Limonene | 8% |
| β-caryophyllene | 4% |
| Total | 100% |

### Example 14

| Sucrose | 50% w/w |
|---|---|
| Maltose | 20% |
| Low-methoxy pectin | 1.0% |
| Carboxymethylcellulose | 10% |
| Gelatin | 4.0% |
| Soybean oil | 2.0% |
| Milk proteins | 1.0% |
| Limonene | 8% |
| β-caryophyllene | 4% |
| Total | 100% |

### Example 15

| Sucrose | 50% w/w |
|---|---|
| Maltose | 20% |
| Low-methoxy pectin | 1.0% |
| Corn syrup | 10% |
| ethyl lineolate | 4.0% |
| Soybean oil | 2.0% |
| Milk proteins | 1.0% |
| Limonene | 8% |
| β-caryophyllene | 4% |
| Total | 100% |

### Example 16

| Sucrose | 50% w/w |
|---|---|
| Maltose | 20% |
| Low-methoxy pectin | 1.0% |
| linolenic acid | 10% |
| Gelatin | 4.0% |
| Soybean oil | 2.0% |
| Milk proteins | 1.0% |
| Limonene | 8% |
| β-caryophyllene | 4% |
| Total | 100% |

### Example 17

| Sucrose | 50% w/w |
|---|---|
| Maltose | 20% |
| Low-methoxy pectin | 1.0% |
| Corn syrup | 10% |
| Gelatin | 4.0% |
| Palm oil | 2.0% |
| Milk proteins | 1.0% |
| Limonene | 8% |
| β-Caryophyllene | 4% |
| Total | 100% |

### Example 18

| Sucrose | 50% w/w |
|---|---|
| Maltose | 20% |
| Low-methoxy pectin | 1.0% |
| Corn syrup | 10% |
| Gelatin | 4.0% |
| Soybean oil | 2.0% |
| Milk proteins | 1.0% |
| Limonene | 8% |
| β-caryophyllene | 4% |
| Total | 100% |

### Example 19. Formulations Having Improved Terpene Recovery

Table 1 reports example data of improved terpene recovery from medicated chews formulated according to methods of the present disclosure.

TABLE 1
Summary of Terpene recovery from Medicated
Chew manufacturing trials.

| | | Processing Step | Terpene in | | Percent |
|---|---|---|---|---|---|
| | Flavor | for Terpene | Formulation (%) | | Re- |
| Formulation | Added | Addition | Target | Recovery | covery |
| ID# RD5594 | Water | Before cook, with | 0.0% | 0.00% | N/A |
| | soluble | pectin slurry | | | |
| ID# RD5595 | Water | Before cook, with | 1.0% | 0.30% | 30% |
| | soluble | pectin slurry | | | |
| ID# RD5596 | Water | Before cook, with | 1.0% | 0.10% | 10% |
| | soluble | pectin slurry | | | |
| ID# RD5597 | Water | Before cook, with | 1.0% | 0.12% | 12% |
| | soluble | pectin slurry | | | |
| ID# RD5598 | Water | Before cook, with | 1.0% | 0.19% | 19% |
| | soluble | pectin slurry | | | |
| ID# 000RD4 | Water | After cook | 1.0% | 0.06% | 6% |
| | soluble | | | | |
| ID# 000RD3 | Water | After cook | 1.0% | 0.04% | 4% |
| | soluble | | | | |
| ID# 000RD1 | Water | After cook | 1.0% | 0.08% | 8% |
| | soluble | | | | |
| ID# 000RD2 | Water | After cook | 1.0% | 0.20% | 20% |
| | soluble | | | | |
| ID# 130189 | Oil | Before cook, with | 1.0% | 0.95% | 95% |
| | Soluble | pectin slurry | | | |
| ID# 130191 | Oil | Before cook, with | 1.0% | 0.97% | 97% |
| | Soluble | pectin slurry | | | |
| ID# 130216 | Water | After cook | 1.0% | 0.48% | 48% |
| | soluble | | | | |
| ID# 130221 | Oil | After cook | 1.0% | 0.90% | 90% |
| | Soluble | | | | |
| ID# 130226 | Oil | After cook | 1.0% | 0.67% | 67% |
| | Soluble | | | | |
| ID#127948 | Oil | Before cook, with | 1.0% | 1.02% | 102% |
| | Soluble | pectin slurry | | | |
| ID#127950 | Oil | Before cook, with | 1.0% | 1.05% | 105% |
| | Soluble | pectin slurry | | | |
| ID#127952 | Oil | Before cook, with | 1.0% | 1.11% | 111% |
| | Soluble | pectin slurry | | | |

### Example 20. Analysis of Terpene Content in Example Chewable Dosage Forms

This Example describes analysis of terpene content in example chewable dosage forms produced according to the methods described in the present disclosure, and in several example commercially available chewable dosage forms from other manufacturers. The example chewable dosage forms analyzed are summarized in Table 2.

TABLE 2
Example chewable dosage forms analyzed for terpene content.

| | Sample | |
|---|---|---|
| | Name | Product |
| Samples from | A | Hi Color Nano Black Currant |
| other | B | Hi Color RSO Pineberry |
| manufacturers | C | Select-Strawberry Peach Classics |
| | D | Watermelon Do Drops |
| | E | Wana Watermelon 10 mg |
| | F | Encore Strawberry Peach 2:1 |
| | G | Encore RSO Grape |
| Samples | PL05 | Pineapple Terpene chews-5 mg THC |
| produced | PL25 | Pineapple Terpene chews-25 mg THC |
| according to | OR05 | Orange Terpene chews-5 mg THC |
| the present | OR25 | Orange Terpene chews-25 mg THC |
| disclosure | LL05 | Lemon Lime Terpene chews-5 mg THC |
| | LL25 | Lemon Lime Terpene chews-25 mg THC |

For each of the samples produced according to the present disclosure, the total terpene input into each formulation (before cooking) was 0.5%.

Each sample in Table 2 was analyzed for terpene content as follows:

Samples were prepared by weighing 2 g of sample into a first 50 mL centrifuge tube. 10 mL water was added, and the tube was shaken on a wrist-action shaker until dissolved (typically 20-30 minutes). 10 mL ethyl acetate (EtOAc) was added, and the tube was vortexed for 5 minutes. The tube was then centrifuged, before the EtOAc layer was withdrawn into a second tube. Another 10 mL EtOAc was added to the first tube, followed by vortexing the tube for 5 minutes, and centrifugation. The EtOAc layer from the first tube was then withdrawn and added to the second tube, to provide a total of 20 mL EtOAc layer collected in the second tube. The second tube was then vortexed briefly, and the 20 mL EtOAc layer was filtered.

The filtered EtOAc layer was analyzed using a gas chromatograph mass spectrometer (Shimadzu TQ8050) operated in single quad MS mode (single ion monitoring quantitative analysis), with liquid injection.

The results of the analysis are shown in Table 3, which reports the analyzed terpene content of each sample as weight/weight percentage (w/w %) for total terpenes, and for each of a number of particular terpenes, as indicated in Table 3. For cells of Table 3 that do not report terpene content, the particular terpene content was below the minimum reporting limit in that sample.

TABLE 3
Terpene content (w/w %) in example chewable dosage forms manufactured according to
the present disclosure, compared to chewable dosage forms from other manufacturers.

| Sample | PL05 | PL25 | OR05 | OR25 | LL05 | LL25 | A | B | C | D | E | F | G |
|---|---|---|---|---|---|---|---|---|---|---|---|---|---|
| Total | 0.247 | 0.275 | 0.337 | 0.394 | 0.447 | 0.681 | 0.000 | 0.005 | 0.014 | 0.022 | 0.003 | 0.020 | 0.014 |
| Terpenes | | | | | | | | | | | | | |
| w/w (%) | | | | | | | | | | | | | |
| Alpha- | | | 0.010 | 0.020 | 0.040 | 0.080 | | | | 0.020 | | | |
| Pinene | | | | | | | | | | | | | |
| w/w (%) | | | | | | | | | | | | | |
| Sabinene | | | | | 0.002 | 0.002 | | | | | | | |
| w/w (%) | | | | | | | | | | | | | |
| beta- | | | 0.006 | 0.008 | 0.040 | 0.074 | | | | | | | |
| Pinene | | | | | | | | | | | | | |
| w/w (%) | | | | | | | | | | | | | |
| beta- | 0.053 | 0.072 | 0.008 | 0.008 | 0.031 | 0.046 | | | | | | | |
| Myrcene | | | | | | | | | | | | | |
| w/w (%) | | | | | | | | | | | | | |
| alpha- | | | | | 0.002 | 0.003 | | | | | | | |
| Terpinene | | | | | | | | | | | | | |
| w/w (%) | | | | | | | | | | | | | |
| Limonene | 0.039 | 0.051 | 0.102 | 0.134 | 0.180 | 0.301 | | | | | | | |
| w/w (%) | | | | | | | | | | | | | |
| Ocimene | | | 0.008 | 0.007 | | | | | | | | | |
| w/w (%) | | | | | | | | | | | | | |
| gamma- | | | | 0.001 | 0.022 | 0.034 | | | | | | | |
| Terpinene | | | | | | | | | | | | | |
| w/w (%) | | | | | | | | | | | | | |
| Terpinolene | | | 0.006 | 0.006 | 0.022 | 0.031 | | | | | | | |
| Linalool | 0.023 | 0.025 | 0.009 | 0.009 | 0.012 | 0.016 | | | 0.004 | | 0.003 | 0.003 | 0.004 |
| w/w (%) | | | | | | | | | | | | | |
| Fenchol | | | | | 0.002 | 0.002 | | | | | | | |
| w/w (%) | | | | | | | | | | | | | |
| Borneol | | | | | 0.001 | 0.001 | | | | | | | |
| w/w (%) | | | | | | | | | | | | | |
| Terpineol | 0.003 | | 0.005 | 0.004 | 0.007 | 0.008 | | | 0.002 | 0.002 | | | 0.002 |
| w/w (%) | | | | | | | | | | | | | |
| beta- | 0.046 | 0.047 | 0.072 | 0.082 | 0.039 | 0.038 | | 0.002 | | | | 0.004 | 0.004 |
| Caryophyllene | | | | | | | | | | | | | |
| w/w (%) | | | | | | | | | | | | | |
| alpha- | 0.025 | 0.025 | 0.016 | 0.017 | 0.017 | 0.016 | | | | | | | |
| Humulene | | | | | | | | | | | | | |
| w/w (%) | | | | | | | | | | | | | |
| Valencene | | | 0.046 | 0.047 | | | | | | | | | |
| w/w (%) | | | | | | | | | | | | | |
| trans- | 0.052 | 0.049 | 0.041 | 0.043 | 0.024 | 0.022 | | | | | | | |
| Nerolidol | | | | | | | | | | | | | |
| w/w (%) | | | | | | | | | | | | | |
| Caryophyllene | 0.006 | 0.006 | 0.008 | 0.008 | 0.006 | 0.007 | | | | | | 0.005 | |
| oxide | | | | | | | | | | | | | |
| w/w (%) | | | | | | | | | | | | | |
| beta- | | | | | | | | | 0.004 | | | 0.004 | |
| Eudesmol | | | | | | | | | | | | | |
| w/w (%) | | | | | | | | | | | | | |
| alpha- | | | | | | | | 0.003 | 0.004 | | | 0.004 | 0.004 |
| Bisabolol | | | | | | | | | | | | | |
| w/w (%) | | | | | | | | | | | | | |

Table 3 clearly shows that the example chewable dosage forms manufactured according to the methods described herein, have much higher terpene content than the example chewable dosage forms from other manufacturers. In fact, in the example dosage forms from other manufacturers, most terpenes were not detectable, and the terpenes that were detectable were only present in very low levels. These results provide further evidence to support the superior utility of the methods described herein for maintaining concentrations of volatile lipophilic compounds in manufacture of dosage forms.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives. The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. When particular values or compositions are provided in the instant disclosure, unless otherwise stated, the meaning of “about” or “approximately” should be assumed to be within an acceptable error range for that particular value or composition.

The term “administering”, “administered” and grammatical variants refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include oral, topical, epidermal or mucosal route of administration, for example, intranasally, vaginally, rectally, sublingually or topically. Administering can also be performed, for example, once, a plurality of times, and/or over one or more extended periods.

The above disclosure contains various examples of stabilized emulsions and dosage forms. Aspects of these various examples may all be combined with one another, even if not expressly combined in the present disclosure, unless they are clearly mutually exclusive.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of providing a dosage form comprising one or more volatile lipophilic compounds, the method comprising:
combining the one or more volatile lipophilic compounds with one or more water-insoluble components to provide a mixture; and
processing the mixture to provide the dosage form, wherein the processing comprises one or more manufacturing methods capable of increasing vapor pressure of the one or more volatile lipophilic compounds when the one or more volatile lipophilic compounds are not combined with the one or more water-insoluble components.

2. The method of claim 1, wherein the one or more volatile lipophilic compounds has a first vapor pressure at a first temperature; the mixture has a second vapor pressure at the first temperature; and the second vapor pressure is lower than the first vapor pressure.

3. The method of claim 1, further comprising:
before the processing, adding one or more surface-active agents and one or more water-soluble components to the mixture; and
emulsifying the mixture comprising the one or more surface-active agents and the one or more water-soluble components to provide an emulsion.

4. The method of claim 3, wherein the one or more volatile lipophilic compounds has a first vapor pressure at a first temperature; the emulsion has a third vapor pressure at the first temperature; and the third vapor pressure is lower than the first vapor pressure.

5. The method of claim 1, wherein the one or more volatile lipophilic compounds comprise one or more essential oils.

6. The method of claim 5, wherein the one or more essential oils are selected from the group consisting of: myrcene, beta-caryophyllene, limonene, linalool, pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, cymene, octanol, isopulegol, cedrene, camphene, geranyl acetate, bergamotene, camphor, pulegone, and any combinations thereof.

7. The method of claim 1, wherein the one or more water-insoluble components comprises one or more oils, fats, waxes, polymers, or any combinations thereof.

8. The method of claim 1, wherein the one or more water-insoluble components are selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether, and any combinations thereof.

9. The method of claim 3, wherein the one or more surface-active agents are selected from the group consisting of: glyceryl monostearate, lecithin, a Tween, a Span, a modified food starch, a monoglyceride, a diglyceride, a cellulose derivative, guar gum, carrageenan, Acacia gum, a hydrocolloid, a fatty ester, a long-chain fatty acid, sodium citrate, pectin, and any combinations thereof.

10. The method of claim 3, wherein the one or more water-soluble components are selected from the group consisting of: gelatin, whey, casein, albumin, pectin, modified food starch, microcrystalline cellulose, hydroxypropyl methylcellulose, hydroxypropyl cellulose, polyvinyl alcohol, polyvinylpolypyrrolidone, beta celluloses, and any combinations thereof.

11. The method of claim 3, wherein the emulsifying is performed using a high-shear mixer.

12. The method of claim 1, wherein the one or more manufacturing methods are selected from the group consisting of: mixing, shearing, granulating, blending, heating, pressurizing, spray-drying, extrusion, spheronization, micro-encapsulation, and any combinations thereof.

13. The method of claim 1, wherein the dosage form is selected from the group consisting of: a solid dosage form, a soft chewable dosage form, a multi-particulate solid, a fast dissolving dosage form, a semi-solid dosage form, and an inhaled dosage form.

14. The method of claim 13, wherein the solid dosage form is a compressed tablet, a lozenge, a troche, a chewable tablet, a capsule, a mini-tablet, or any combinations thereof.

15. The method of claim 13, wherein the soft chewable dosage form is a gummy, a gum, or a combination thereof.

16. The method of claim 13, wherein the multi-particulate solid is a powder, a sprinkle, a suspension, a bulk granulation, or any combinations thereof.

17. The method of claim 13, wherein the fast dissolving dosage form is a fast dissolving tablet, a fast-dissolving film, a sublingual tablet, a sublingual film, a buccal tablet, a buccal film, or any combinations thereof.

18. The method of claim 13, wherein the semi-solid dosage form is a cream, an ointment, a tincture, a spray, a suppository, a liniment, a lotion, a gel, a paste, a balm, a salve, an infused bandage, an infused patch, or any combinations thereof.

19. The method of claim 13, wherein the inhaled dosage form is a dosage form administrable with an aerosol inhaler, a vape pen, a vape device, and aerosol forming device, a dry powder inhaler, a nebulizer, or any combinations thereof.

20. The method of claim 13, wherein the inhaled dosage form is administrable by smoking and inhaling, wherein the smoking is by combustion, electronic means, or a combination thereof.

21. The method of claim 15, wherein the gum comprises one or more gum-forming components.

22. The method of claim 21, wherein the one or more gum-forming components are selected from the group consisting of: gelatin, xanthan gum, guar gum, pectin, modified starch, and any combinations thereof.

23. The method of claim 21, wherein the gum is formed using one or more methods selected from: tableting, molding, rolling, depositing, slabbing, or any combinations thereof.

24. The method of claim 3, further comprising:
after the emulsifying, covering the emulsion with a layer of one or more water-insoluble components.

25. The method of claim 24, wherein the one or more water-insoluble components comprises one or more oils, fats, waxes, polymers, or any combinations thereof.

26. The method of claim 24, wherein the one or more water-insoluble components are selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether and any combinations thereof.

27. The method of claim 3, wherein the one or more surface-active agents comprises one or more proteinaceous materials.

28. The method of claim 27, wherein the one or more proteinaceous materials are selected from the group consisting of: albumin, gelatin, whey protein, a caseinate, a plant-based protein, and any combinations thereof.

29. A dosage form produced by the method according to any one of the preceding claims.

30. A composition comprising:
a mixture comprising: (i) one or more volatile lipophilic compounds and (ii) one or more water-insoluble components;
wherein the one or more volatile lipophilic compounds are present in an amount from 0.001% to 15% by weight of the composition.

31. The composition of claim 30, wherein the one or more volatile lipophilic compounds has a first vapor pressure at a first temperature; the mixture has a second vapor pressure at the first temperature; and the second vapor pressure is lower than the first vapor pressure.

32. The composition of claim 30, wherein the composition comprises an emulsion of the mixture and one or more surface-active agents and one or more water-soluble components.

33. The composition of claim 32, wherein the emulsion has a third vapor pressure at the first temperature, and the third vapor pressure is lower than the first vapor pressure.

34. The composition of claim 30, wherein the one or more volatile lipophilic compounds comprises one or more essential oils.

35. The composition of claim 34, wherein the one or more essential oils are selected from the group consisting of: myrcene, beta-caryophyllene, limonene, linalool, alpha-pinene, beta-pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, cymene, octanol, isopulegol, cedrene, camphene, geranyl acetate, bergamotene, camphor, pulegone, and any combinations thereof.

36. The composition of claim 30, wherein the one or more water-insoluble components comprises one or more oils, fats, waxes, polymers, or any combinations thereof.

37. The composition of claim 30, wherein the one or more water-insoluble components are selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether, and any combinations thereof.

38. The composition of claim 32, wherein the one or more surface-active agents are selected from the group consisting of: glyceryl monostearate, lecithin, a Tween, a Span, a modified food starch, a monoglyceride, a diglyceride, a cellulose derivative, guar gum, carrageenan, Acacia gum, a hydrocolloid, a fatty ester, a long-chain fatty acid, sodium citrate, pectin, and any combinations thereof.

39. The composition of claim 32, wherein the one or more water-soluble components are selected from the group consisting of: gelatin, whey, casein, albumin, pectin, modified food starch, microcrystalline cellulose, hydroxypropyl methylcellulose, hydroxypropyl cellulose, polyvinyl alcohol, polyvinylpolypyrrolidone, beta celluloses, and any combinations thereof.

40. The composition of claim 30, formulated as a dosage form selected from the group consisting of: a solid dosage form, a soft chewable dosage form, a multi-particulate solid, a fast dissolving dosage form, a semi-solid dosage form, and an inhaled dosage form.

41. The composition of claim 40, wherein the solid dosage form is a compressed tablet, a lozenge, a troche, a chewable tablet, a capsule, a mini-tablet, or any combinations thereof.

42. The composition of claim 40, wherein the soft chewable dosage form is a gummy, a gum, or a combination thereof.

43. The composition of claim 40, wherein the multi-particulate solid is a powder, a sprinkle, a suspension, a bulk granulation, or any combinations thereof.

44. The composition of claim 40, wherein the fast dissolving dosage form is a fast dissolving tablet, a fast-dissolving film, a sublingual tablet, a sublingual film, a buccal tablet, a buccal film, or any combinations thereof.

45. The composition of claim 40, wherein the semi-solid dosage form is a cream, an ointment, a tincture, a spray, a suppository, a liniment, a lotion, a gel, a paste, a balm, a salve, an infused bandage, an infused patch, or any combinations thereof.

46. The composition of claim 40, wherein the inhaled dosage form is a dosage form administrable with an aerosol inhaler, a vape pen, a vape device, and aerosol forming device, a dry powder inhaler, a nebulizer, or any combinations thereof.

47. The composition of claim 40, wherein the inhaled dosage form is administrable by smoking and inhaling, wherein the smoking is by combustion, electronic means, or a combination thereof.

48. The composition of claim 42, wherein the gum comprises one or more gum-forming components.

49. The composition of claim 48, wherein the one or more gum-forming components are selected from the group consisting of: gelatin, xanthan gum, guar gum, pectin, modified starch, and any combinations thereof.

50. The composition of claim 32, further comprising a layer of one or more water-insoluble components disposed around the emulsion.

51. The composition of claim 50, wherein the one or more water-insoluble components comprises one or more oils, fats, waxes, polymers, or any combinations thereof.

52. The composition of claim 50, wherein the one or more water-insoluble components are selected from the group consisting of: coconut oil, olive oil, a vegetable oil, canola oil, soybean oil, corn oil, avocado oil, palm oil, palm kernel oil, carnauba wax, white wax, bees wax, lauroyl polyoxyl-32 glycerides, polyoxyl-32 stearate, stearoyl polyoxyl-32 glycerides, glyceryl monocaprylocaprate, glyceryl dicaprylocaprate, propylene glycol dicaprolate/dicaprate, oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate, glyceryl monooleate, polyglyceryl-3 dioleate, diethylene glycol, monoethyl ether and any combinations thereof.

53. The composition of claim 32, wherein the one or more surface-active agents comprises one or more proteinaceous materials.

54. The composition of claim 53, wherein the one or more proteinaceous materials are selected from the group consisting of: albumin, gelatin, whey protein, a caseinate, a plant-based protein, and any combinations thereof.

55. The composition of claim 30, wherein a total amount of the volatile lipophilic compounds is present in an amount from 0.001% to 15% by weight of the composition.

56. The composition of claim 30, wherein at least one of the volatile lipophilic compounds are present in an amount from 0.001% to 15% by weight of the composition.

57. The method of claim 1, wherein the one or more volatile lipophilic compounds comprises one or more terpenes.

58. The composition of claim 30, wherein the one or more volatile lipophilic compounds comprises one or more terpenes.
